(19) United States
(12) Patent Application Publication
(10) Pub. No.: US 2006/0148681 A1
(43) Pub. Date: Jul. 6, 2006

(54) Method for treating tumor and/or preventing tumor metastasis and relapse

(75) Inventors: Huan-Yao Lei, Tainan City (TW); Chih-Peng Chang, Yuanlin Township (TW); Wei-Chun Cheng, Hsinchu City (TW)

Correspondence Address: LADAS & PARRY, 26 WEST 61ST STREET, NEW YORK, NY, 10023, US

(73) Assignee: NATIONAL CHENG KUNG UNIVERSITY

(21) Appl. No.: 11/322,094

(22) Filed: Dec. 29, 2005

(30) Foreign Application Priority Data
Dec. 31, 2004 (TW) 093141805

(51) Int. Cl. A61K 38/16

(52) U.S. Cl. 514/8

(57) ABSTRACT

The present invention provides a method for treating tumor and/or preventing tumor metastasis and relapse comprising administrating a subject with a therapeutically effective amount of lectin capable of binding to the tumor and having tumor cytotoxicity and/or lymphocyte activation activity.

### BACKGROUND OF THE INVENTION

1. Field of the Invention

The invention relates to a method for treating tumor and/or preventing tumor metastasis and relapse. More particularly, the invention relates to the use of lectin in treating tumor and/or preventing tumor metastasis and relapse.

2. Description of the Related Art

Lectin is a glycoprotein capable of binding to certain monosaccharide molecules, which can agglutinate cells and bind to specific carbohydrates or carbohydrate-containing compounds. Lectin widely exists in nature. Lectin and its analogues can be found in plants, microorganisms and animals. In particular, plant seeds are rich in lectin. Lectin is considered a substance guarding plants from environmental toxins.

Lectin was originally named because of its ability to agglutinate red blood cells. Due to its carbohydrate-binding moiety, lectin has the ability to specifically bind to carbohydrate molecules, such as mannose, glucose, N-acetyl glucosamine, and galactose. Therefore, it is usually used in researches concerning glycoproteins on cell surfaces.

In immunology, lectin is a potent mitogenic factor for stimulating lymphocyte mitosis. In another aspect, human endogenous lectin is also used as the first-line defense against microbial agents or as a transmitter of cellular communication.

Because the degree of glycosylation relates to the malignancy and metastatic activities in the development of cancerous cells, extracts derived from plants are often used as an adjuvant of injections for treating cancers. The active compounds in the extracts are usually lectin with cytotoxicity and immunostimulating effect. Among such extracts, the lectin from a mistletoe extract, which specifically binds to galactose, has slighter toxicity and thus has been adopted for alternative tumor therapy in Europe (Stauder, H. et. al., Onkologie 25, 374-380, 2002; Schumacher et. al., Anticancer Res. 23, 5081-5087, 2003).

Concanavalin A (Con A) is a lectin derived from Canavalia ensiformis. Concanavalin A is a T-cell mitogen and has been used to induce hepatitis in mice through triggering NK T cell and subsequent activation of CD⁴⁺ T cells (Tiegs, G. et. al., A. J. Clin. Invest. 90, 196-203, 1992; Kaneko et. al., J. Exp. Med. 191, 105-114, 2000). Furthermore, Concanavalin A has also been reported to inhibit colorectum cancer cell line growth when co-cultured with the cell line (Kiss R. et. al., Gut. 40(2):253-61, 1997).

Pisum Sativum Agglutinin (PSA), which is derived from Pisum sativum, is capable of binding to glucose and mannose.

Phaseolus vulgaris leucoagglutinin (PHA-L), which is derived from Phaseolus vulgaris, has been reported to inhibit the growth of non Hodgkin's lymphoma or Krebs II lymphosarcoma when co-cultured with these cell lines (Pryme I F. et. al., Cancer Letters. 146(1):87-91, 1999; Pryme I F. et. al., Journal of Experimental Therapeutics & Oncology. 1(5):273-7, 1996; Pryme I F et. al., Cancer Letters. 76(2-3):133-7, 1994). However, it is also reported that the administration of Phaseolus vulgaris leucoagglutinin alone in vivo fails to eliminate plasmacytoma in mice (Pryme I F. et al., Cancer Letters. 103(2):151-5, 1996).

Tumor growth involves numerous mechanisms and are complicated. A method for effectively treating tumor, preventing tumor metastasis and inhibiting tumor relapse is needed in this field.

### SUMMARY OF THE INVENTION

One object of the invention is to provide a method for treating tumor.

Another object of the invention is to provide a method for preventing tumor metastasis.

Still another object of the invention is to provide a method for inhibiting tumor relapse.

The objects mentioned above can be achieved by administrating a subject with a therapeutically effective amount of lectin capable of binding to the tumor and having tumor cytotoxicity and/or lymphocyte activation activity. Preferably, the lectin is selected from the group consisting of Concanavalin A, Pisum Sativum Agglutinin and Phaseolus vulgaris leucoagglutinin.

The invention also provides a composition for treating tumor and/or preventing tumor metastasis and relapse comprising a therapeutically effective amount of lectin capable of binding to the tumor and having tumor cytotoxicity and/or lymphocyte activation activity.

### BRIEF DESCRIPTION OF THE DRAWINGS

FIG. 1. Results of binding assay of lectin to tumor cell lines and splenocytes. ML-1₄ₐ (a), CT-26 (b), Huh-7 (c), and splenocytes (d) at 1×10⁵/mL were incubated with 5 μg/mL of lectin-conjugated fluorescein for 30 m at 37° C. The binding was detected with FACSCalibur.

FIG. 2. Results of apoptosis assay of lectins. ML-1₄ₐ (a), CT-26 (b), Huh-7 (c) at 1×10⁵/mL were co-cultured with Con A, LCA, WGA, RCA-1 or PHA-E at various concentrations for 24 h. The apoptosis was quantified using Annexin V with FACSCalibur.

FIG. 3. Mitogenic activity induced by lectins on mouse splenocytes. Murine splenocytes (2×10⁵/mL) were cultured with various concentrations of Con A, LCA, PSA, WGA, RCA-1, GSL-1, DBA, SJA, PNA, SBA, PHA-L or PHA-E for 72 h. The proliferation was detected by H³-thymidine incorporation.

FIG. 4. Results of binding assay of Con A to mannose residues of hepatoma cells and apoptosis induction. (a) ML-1₄ₐ, CT-26, Huh-7, and HepG2 stained with Con A-FITC were detected by FACScalibur. Methyl-αD-mannopyranoside was used to block the specific binding. Bound Con A inhibited cell growth by inducing apoptosis. (b) ML-1₄ₐ, CT-26, Huh-7 and HepG2 cells were incubated with various concentrations of Con A for 72 h, and the viable cell numbers were counted. (c) Con A-induced apoptosis were determined by Annexin-V/PI staining. ML-1₄ₐ, CT-26, and HepG2 cells were incubated with 50 μg/mL of Con A.

FIG. 5. Results of Con A inhibiting liver tumor nodule formation in vivo. Intra-spleen inoculation of ML-1₄ₐ established tumor nodule formation in the liver of BALB/c mice. Con A was administrated intravenously two to five times 3-day intervals. On day 30 or 32, the number and sizes of the tumor nodules in the liver were determined. (a-c). Con A caused hepatoma regression in the liver of one-week-old, hepatoma-bearing mice. Con A (7.5 mg/kg) given twice beginning at day 7 inhibited tumor nodule formation (a&b, n=7), and prolonged the survival of the tumor-bearing mice (c, n=10). (d-e). Con A partially inhibited the liver tumor nodule formation and prolonged the survival of two-week-old, hepatoma-bearing mice. Con A (7.5, 10, 15 mg/kg) given twice beginning at day 14 partially inhibited tumor nodule formation (d, n=7). Con A (20 mg/kg) given five times prolonged the survival of the tumor-bearing mice (e, n=10). (f-g) Con A partially inhibited the liver tumor formation and prolonged the survival of three-week-old, hepatoma-bearing mice. Con A (10, 15 or 20 mg/kg) given four times beginning at day 21 partially inhibited tumor formation (f, n=7). Con A (20 mg/kg) given five times prolonged the survival of the tumor-bearing mice (g, n=10). Two or three experiments of each type were repeated with the same results. * p<0.05.

FIG. 6. Histopathology of the liver of hepatoma-one-week-old, bearing-mice treated with Con A. Intra-spleen inoculation of ML-1₄ₐ established tumor nodule formation in the liver of BALB/c mice. Con A (7.5 mg/kg) was administrated intravenously on day 7, and the liver was removed 2 days post Con A treatment and stained with H&E. (a) Naive. (b) PBS-treated. (c&d) Con A-treated. The arrow points to lymphocyte infiltrations.

FIG. 7. Results of Con A-mediated liver tumor nodule inhibition. The effect of CD4⁺ or CD8⁺ depletion on tumor nodule formation was studied. The CD4⁺ or CD8⁺ lymphocytes were depleted by the intraperitoneal injection of 100 μL ascites of GK1.5 (anti-CD4) or 2.43 (anti-CD8) 7 days before tumor inoculation and every 7 days thereafter during the experimental period.

FIG. 8. Results of oral feeding of Con A for preventing liver tumor nodule formation in BALB/c mice. Groups of 10 BABL/c mice were inoculated of 1×10⁶ ML-1₄ₐ cells by intrasplenic injection. The mice were orally fed with PBS (control) or Con A (300, 500 mg/kg) everyday from one day before inoculation and thereafter. On day 21, the number and size of tumor nodules were determined. * p<0.05 vs. PBS-treated control.

FIG. 9. Results of Con A preventing the hepatic metastasis of colon cancer cells. Intra-spleen inoculation of CT-26 established hepatic metastatic tumor formation in BALB/c mice. Con A (7.5, 10 mg/kg) given four times beginning on day 2 suppressed the tumor nodule formation in the liver on day 21. Two or three experiments of each type were repeated with the same results. *p<0.05.

FIG. 10. Results of resistance of the same tumor challenge in Con A-cured mice. The tumor-free mice (˜30%) post Con A-treatment mentioned above were inoculated subcutaneously dorsally with ML-1₄ₐ or CT-26 cells two months later. The tumor sizes were determined.

FIG. 11. Results of Con A inhibiting liver tumor nodule formation in SCID mice. Intra-spleen inoculation of ML-14a established tumor nodule formation in the liver of SCID mice. Con A (10, 15, 20 mg/kg) given twice beginning on day 7 inhibited tumor nodule formation (n=7). On day 21, the number and sizes of the tumor nodules in the liver were determined. The experiment was repeated twice with the same results. * p<0.05.

FIG. 12. Results of lectin PSA, PHA-L (a) or LCA (b) inhibiting liver tumor nodule formation in vivo. Intra-spleen inoculation of ML-1₄ₐ established tumor nodule formation in the liver of BALB/c mice. PSA or PHA-L (10, 15 mg/kg) given twice beginning on day 7 inhibited tumor nodule formation (n=7). LCA (10, 15, 20 mg/kg) given twice beginning on day 2 inhibited tumor nodule formation (n=7). On day 21, the number and sizes of the tumor nodules in the liver were determined. The experiments were repeated twice with the same results. * p<0.05.

FIG. 13. The immunofluorescent staining of Con A (a), PSA (b) and LCA (c) to human hepatoma tissues. The paraffin-fixed human HCC tissue and the normal part of the liver of one HCC patient were stained with 20 pg/mL Con A-FITC, LCA-FITC or PSA-FITC at 4° C. overnight. Hoechst was used as the nucleus staining. Methyl-αD-mannopyranoside (0.5 M) was included as a specific blocker. N, normal part; T, tumor part.

### DETAILED DESCRIPTION OF THE INVENTION

The invention provides a method for treating tumor in a subject comprising administrating to the subject a therapeutically effective amount of lectin capable of binding to the tumor and having tumor cytotoxicity and/or lymphocyte activation activity. Preferably, the lectin is selected from the group consisting of Concanavalin A, Pisum Sativum Agglutinin and Phaseolus vulgaris leucoagglutinin.

According to the invention, the lectin capable of binding to the tumor and having tumor cytotoxicity and/or lymphocyte activation activity successfully treats tumor and eliminates the tumor size in a hepatic metastatic colon cancer cell-bearing animal. It is noted that the therapeutic effect on large tumors is particularly significant.

The lectin capable of binding to the tumor and having tumor cytotoxicity and/or lymphocyte activation activity can be screened by methods well known to persons of ordinary skill in this field.

In one embodiment of the invention, lectins capable of binding to tumor cells are first screened. For example, a reporter is utilized for monitoring if the lectins to be detected bind to the tumor; preferably an enzyme-linked immunosorbent assay system is applied. The method is described in Republic of China Patent Application No. 093135900 (filed on Nov. 22, 2004. The corresponding U.S. application was filed on Nov. 21, 2005.) The specification of the application is incorporated herein as a reference.

While not wishing to be limited by theory, it is believed that the affinity between the lectin and the tumor is higher than that between the lectin and a normal cell, because the degree of glycosylation on the tumor surface differs from that on the normal cell in the process of cancerous cell development. Given the above, using lectin in treating tumor according to the invention is highly specific.

The lectin having tumor cytotoxicity can be screened by methods well known to persons having ordinary skill in this field; such as co-culturing the lectin to be detected with the tumor cell and monitoring the growth condition of the tumor cell. Preferably, the lectin with the ability to induce tumor apoptosis is screened. There are several commercially available kits for such screening, e.g., Annexin V-PI kit, BioVision®, Mountain View, Calif.

The lectin having lymphocyte activation activity can be screened by methods well known to persons of ordinary skill in this field, such as co-culturing the lectin to be detected with the lymphocytes and monitoring the growth condition of the lymphocytes.

It was unexpectedly found that the lectin capable of binding to the tumor and having tumor cytotoxicity and/or lymphocyte activation activity not only controls and reduces the size of primary tumors by inducing tumor apoptosis and activating lymphocytes, but also eliminates tumors and decreases the chance of metastasis. Furthermore, the lectin capable of binding to the tumor and having tumor cytotoxicity and/or lymphocyte activation activity can also destroy metastatic tumors when the tumors are small and thus prevent metastasis.

Accordingly, the invention also provides a method for preventing tumor metastasis in a subject comprising administrating to the subject a therapeutically effective amount of lectin capable of binding to the tumor and having tumor cytotoxicity and/or lymphocyte activation activity. Preferably, the lectin is selected from the group consisting of Concanavalin A, Pisum Sativum Agglutinin and Phaseolus vulgaris leucoagglutinin.

It was also unexpectedly found that the lectin capable of binding to the tumor and having tumor cytotoxicity and/or lymphocyte activation activity not only treats tumors by inducing tumor apoptosis and activating lymphocytes, but also establishes immune memory by activating lymphocytes. After a tumor is cured, the immune memory protects the subject from suffering from the same tumor and therefore inhibits tumor relapse.

Accordingly, the invention also provides a method for inhibiting tumor relapse in a subject comprising administrating to the subject with a therapeutically effective amount of lectin capable of binding to the tumor and having tumor cytotoxicity and/or lymphocyte activation activity. Preferably, the lectin is selected from the group consisting of Concanavalin A, Pisum Sativum Agglutinin and Phaseolus vulgaris leucoagglutinin.

The method according to the invention is suitable for treating various tumor cells, including but not limited to hepatoma cells, both primary and metastatic hepatoma cells; especially hepatoma cells transferred from colon cancer cells.

In an animal model according to the invention, the method according to the invention suppresses hepatoma nodule formation, inhibits hepatoma relapse and prevents hepatoma metastasis. In normal liver tissue, the lectin according to the invention stimulates lymphocyte infiltrating into the liver. Cytokines produced by these T lymphocytes cause damage to hepatocyte cells and lead to hepatitis in a normal mouse. To the contrary, the lectin according to the invention directly causes hepatoma apoptosis in a hepatoma-bearing mouse, and the inflammation caused by the infiltrated lymphocytes also stimulates immune response which also kills the hepatoma cells. After the tumor is eradicated, the cured mouse acquires an immune memory with respect to the hepatoma cells which can no longer grow in said mouse.

Preferably, the lectin according to the invention is Concanavalin A.

The present invention also provides a composition for treating tumor and/or preventing tumor metastasis and relapse comprising a therapeutically effective amount of lectin capable of binding to the tumor and having tumor cytotoxicity and/or lymphocyte activation activity. Preferably, the composition is a pharmaceutical composition, which can be in any form known in the art, including but not limited to an injection, an oral tablet, a sublingual or buccal tablet, an oral solution, or syrup. In addition to the lectin as the active substance, the composition may further comprise an adjuvant, an excipient or a carrier necessary for preparing a pharmaceutical composition. The method for preparing the pharmaceutical composition according to the invention and the components other than the active compound can be ascertained by persons of ordinary skills in the art according to the disclosure of the invention.

In another aspect, the composition according to the invention is a food composition such as a health food or a function food.

The composition according to the invention can be administered by methods well known to persons of ordinary skills in the art. For example, the pharmaceutical composition according to the invention in the form of an injection can be injected directly to the tumor, e.g., injected directly to the tumor cells or tumor tissues which allows the lectin to trigger tumor apoptosis and treat tumor, prevent metastasis and/or inhibit relapse through other mechanisms.

In addition, the composition according to the invention can be orally administrated. Through the transportation of the digest and circular systems, the composition according to the invention is delivered to the tumor and thereafter treats tumor, prevents metastasis and/or inhibits relapse. Taking hepatoma cells as an example, the oral composition is delivered to the liver from the stomach through the hepatic portal vein and has a chance to contact the hepatoma cells and hence inhibit hepatoma growth.

The invention further provides use of lectin for manufacturing a drug for treating tumor and/or preventing tumor metastasis and relapse, wherein the lectin is capable of binding to the tumor and having tumor cytotoxicity and/or lymphocyte activation activity. Preferably, the lectin is selected from the group consisting of Concanavalin A, Pisum Sativum Agglutinin and Phaseolus vulgaris leucoagglutinin.

The following Examples are given for the purpose of illustration only and are not intended to limit the scope of the present invention.

### EXAMPLE 1

### Screening Lectin Capable of Binding to Tumor Cells and Lymphocytes and Inducing Their Apoptosis and Proliferation, Respectively

Cell lines and mice. BABL/c hepatoma cell line, ML-1, was kindly provided by Dr. CP Hu (Department of Medical Research, Veterans General Hospital). ML-1₄ₐ cells were adapted from ML-1 cells in BABL/c mice for four generations to increase their tumorogenesity in the liver. Human hepatoma cell lines (Huh-7 and HepG2) were obtained from the Cell Collection and Research Center (CCRC, Hsin-Chu, Taiwan) while murine colon cancer cell line CT-26 was obtained from American Type Culture Collection (Manassas, Va.). All cell lines were cultured in DMEM (Gibco®, Grand Island, N.Y.) supplemented with 10% FBS, L-glutamine and penicillin-streptomycin. BABL/c mice (male, 8-10 weeks old) were purchased from National Laboratory Animal Center (Taipei, Taiwan). The animals were raised and cared for according to the guidelines set up by the National Science Council, ROC. The mouse experiments were approved by the institutional animal care and use committee.

Lectin binding assay. ML-1₄ₐ, CT-26, Huh-7 and murine splenocytes (1×10⁵) were suspended in staining buffer (DMEM containing 2% FBS and 0.1% NaN₃) and co-incubated with 5 μg/mL of fluorescein-conjugated lectin (Vector®, Burlingame, Calif.) for 30 minutes at 37° C. The binding capacity of lectin to cells was detected by flow cytometry.

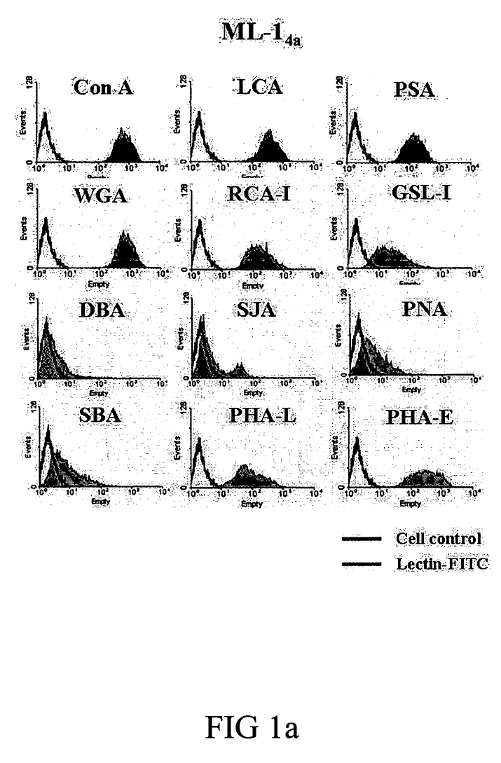

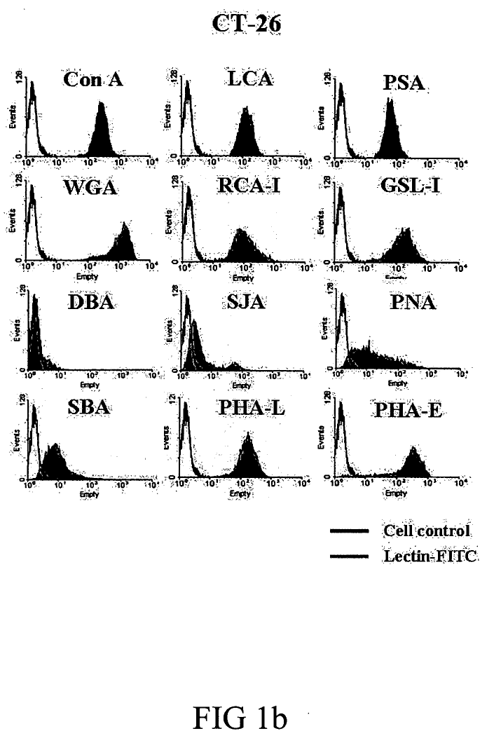

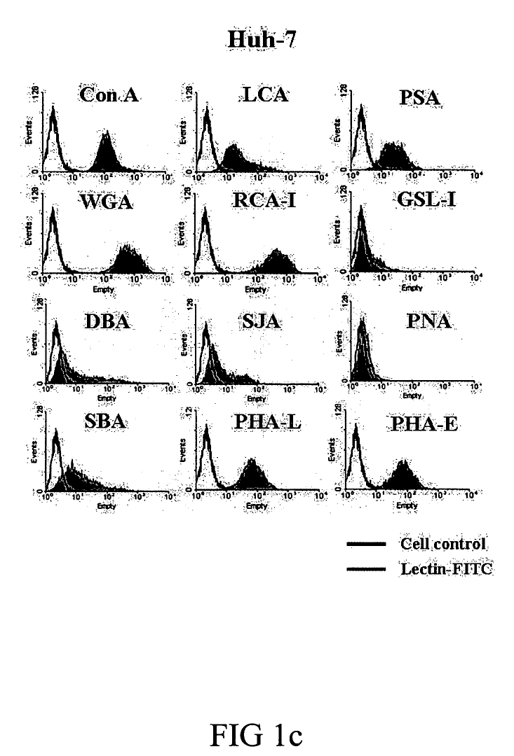

Result: FITC-lectin binding to murine hepatoma cell line ML-1₄ₐ, murine colon cancer cell line CT-26, and human hepatoma cell line Huh-7 were analyzed with the flow cytometry. The results showed that lectins such as Con A, LCA, PSA, WGA, RCA-1, GSL-1, PHA-L, and PHA-E can bind to these tumor cell lines with different affinity (FIGS. 1a, 1b, and 1c). The lectin binding intensity with different tumor cells varied slightly, indicating that the degree of glycosylation of the different tumor cells varied. The lectin binding to murine splenocytes was also tested. The fluorescent intensity detected on lymphocytes was greater than that on tumor cell lines (FIG. 1d), suggesting there was more carbohydrate moiety on lymphocytes.

Apoptosis assay. ML-1₄ₐ, CT-26, and Huh-7 were harvested by trypsinization and seeded 1×10⁵ cells into each well of 12-well plates for 2 hours. Different concentrations of lectins were added into the plates that contained tumor cells. Cells were harvested 24 h later and the apoptosis was quantified by flow cytometry, using the Annexin V-PI kit (BioVision®, Mountain View, Calif.).

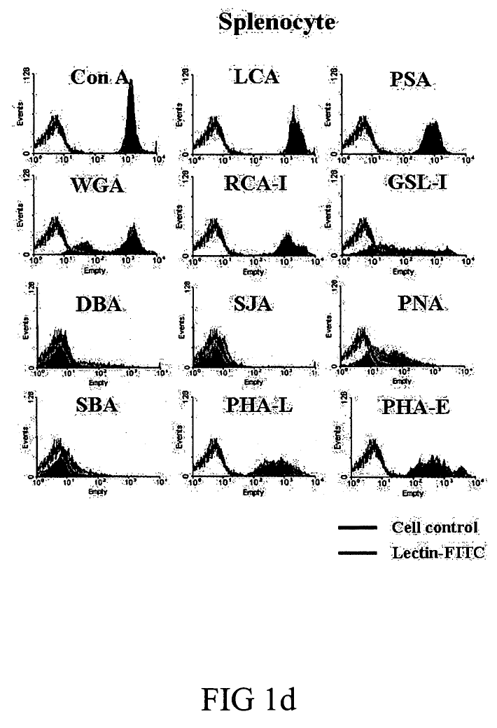

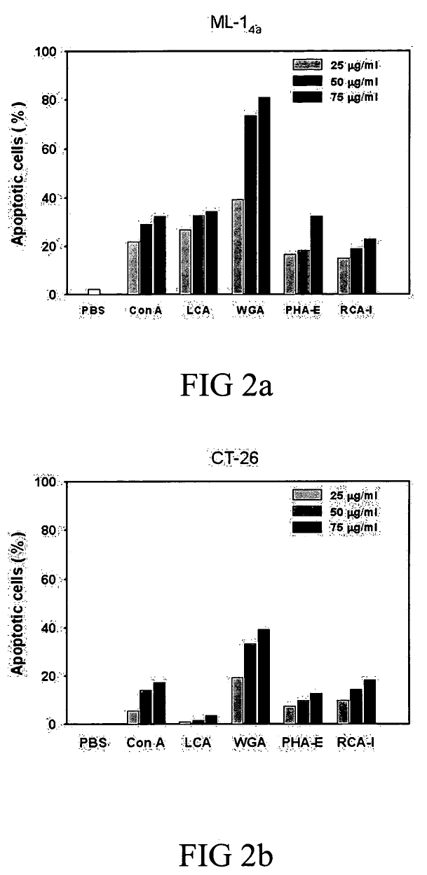

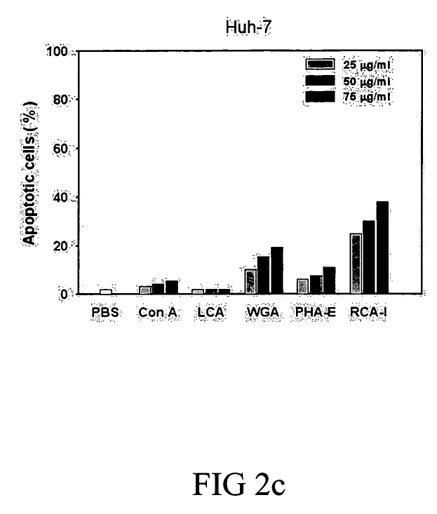

Result: In FIG. 2, Con A, LCA, WGA, PHA-E, or RCA-I could induce tumor cell (ML-1₄ₐ, CT-26, and Huh-7) apoptosis dose-dependently, although the sensitivity of various tumor cells was different, depending on the lectins used.

Mitogenic activities of lectins to splenocytes. BABL/c mice (male, 8-10 weeks old) were purchased from National Laboratory Animal Center (Taipei, Taiwan), and maintained in the pathogen-free facility of the Animal Laboratory of National Cheng Kung University. The splenocytes were isolated from the spleen following normal procedure. Then, 2×10⁵ lymphocytes were stimulated with various lectins in different concentrations for 72 h. Splenocyte proliferation was detected by H³-thymidine incorporation.

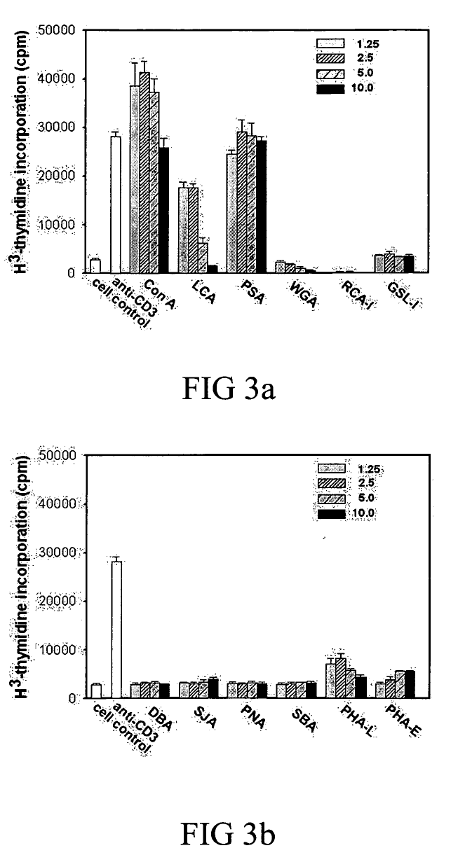

Result: Referring to FIG. 3, Con A, LCA, PSA, and PHA were mitogenic to murine splenocytes while WGA and RCA-1 were toxic to lymphocytes. The dose necessary for lymphocyte proliferation was lower than that for tumor cell apoptosis. Lymphocytes are thus more sensitive than tumor cells probably because of the greater carbohydrate moiety on lymphocytes.

### EXAMPLE 2

### Binding Assay of Con A to Mannose Residues of the Hepatoma Cells and Lymphocytes and Apoptosis and Proliferation Induction, Respectively

Lectin binding assay. ML-1₄ₐ, CT-26, Huh-7, HepG2 and mouse splenocytes (1×10⁵) were suspended in staining buffer (DMEM containing 2% FBS and 0.1% NaN₃) and co-incubated with 5 μg/mL of FITC-conjugated Con A (Vector®, Burlingame, Calif.) for 30 minutes at 37° C. The binding of Con A to cells was detected by flow cytometry. Methyl-αD-mannopyranoside (0.5 M) was used to block the specific binding.

Inhibition of hepatoma cell growth and induction of apoptosis by Con A. ML-1₄ₐ, CT-26, Huh-7 and HepG2 cells harvested by trypsinization were seeded at 1×10⁵ cells into each well of 12-well plates at 37° C. overnight. Different concentrations of Con A were incubated with tumor cells for 72 h. After incubation, cell growth was measured by counting viable cells with Eosin Y exclusion staining. For apoptotic assay, cells harvested at various times and the apoptosis was quantified by flow cytometry using the Annexin V-PI kit.

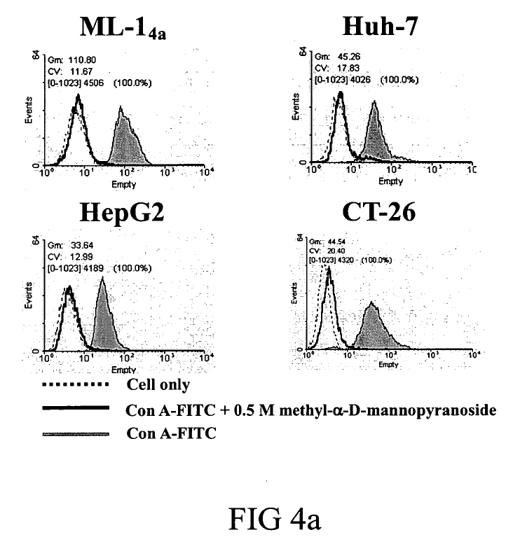

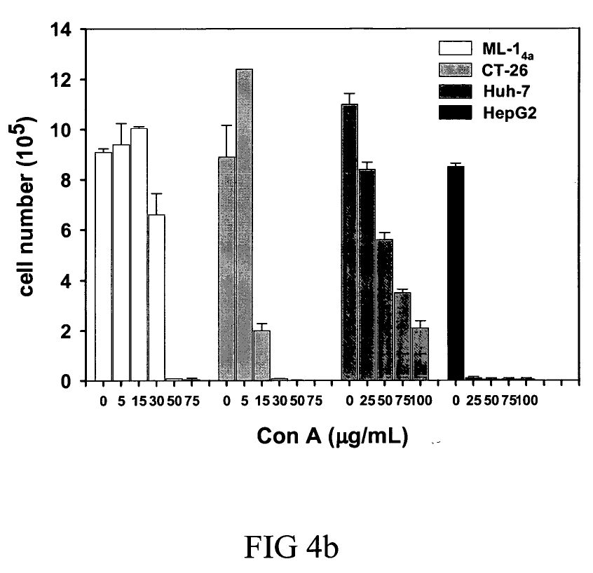

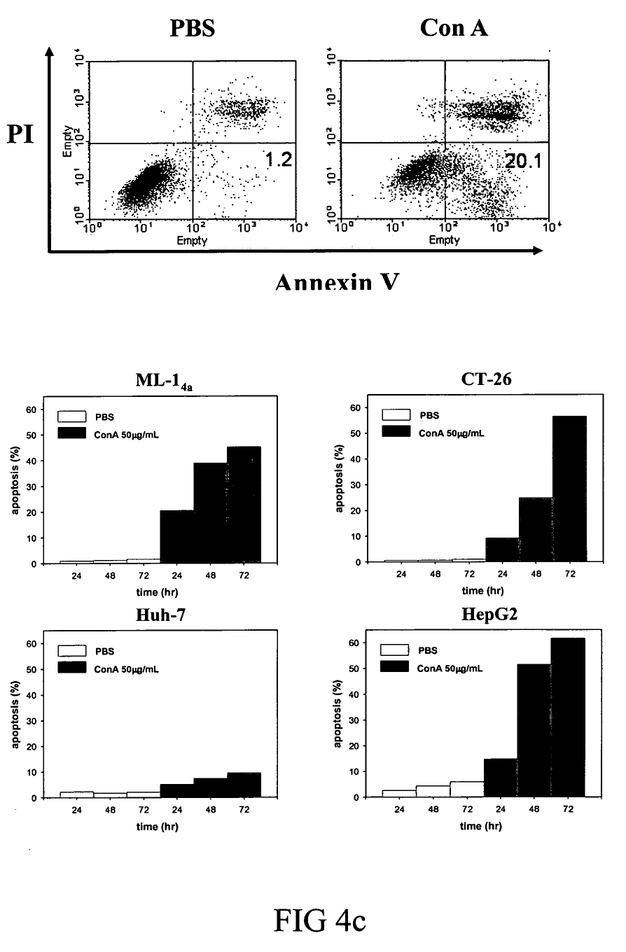

Result: Con A was shown to bind to various hepatoma cell lines in a mannose-specific manner, which was blocked by methyl-αD-mannopyranoside (FIG. 4a). The binding of Con A to ML-1₄ₐ, CT-26, Huh-7 and HepG2 hepatoma cell lines inhibited their growth in a dose-dependent manner (FIG. 4b). This growth inhibition was due to the induction of apoptosis as increase of Annexin-V binding to cell after Con A treatment (FIG. 4c). The sensitivity to Con A-induced cell growth inhibition varied slightly among 4 tumor cell lines: HepG2 was most sensitive, followed by CT-26, ML-1₄ₐ and Huh-7. The IC₅₀ of Con A for HepG2, CT-26, ML-1₄ₐ and Huh-7 were 5, 10, 30 and 50 μg/mL, respectively.

### EXAMPLE 3

### Con A Inhibited Liver Tumor Nodule Formation in vivo

Murine in situ hepatoma model. A murine in situ hepatoma model was set up by intrasplenic injection of 1×10⁶ viable ML-1₄ₐ cells in 0.1 mL of DMEM into anesthetized mice (Pentobarbital, 50 mg/kg i.p.). The ML-1₄ₐ first colonized in the spleen, and then migrated into the liver forming liver nodules of varied size and number beginning 1 week after injection. 30 days post intrasplenic injection, the livers of hepatoma-bearing mice were removed to determine the number and size of the tumors. If the liver tumor nodules were uncountable, instead the liver weight was measured to evaluate the anti-tumor effect. The extent of liver injury was assessed by determination of serum alanine aminotransferase (ALT), and serum asparate aminotransferase (AST) activities. In some experiments, mouse livers were removed and fixed with 3.7% formaldehyde, and the tissue sections were stained with hematoxylin and eosin Y. In the in vivo depletion of CD4⁺ or CD8⁺ lymphocytes, 100 μL ascites of GK1.5 (anti-CD4) or 2.43 (anti-CD8) were injected intraperitoneally 7 days before tumor inoculation and every 7 days thereafter during the experimental period. Depletion was confirmed by FACS analysis of peripheral blood lymphocytes to be >95%.

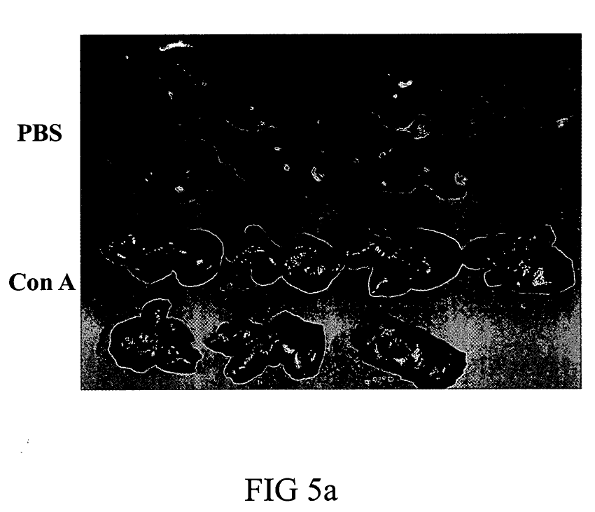

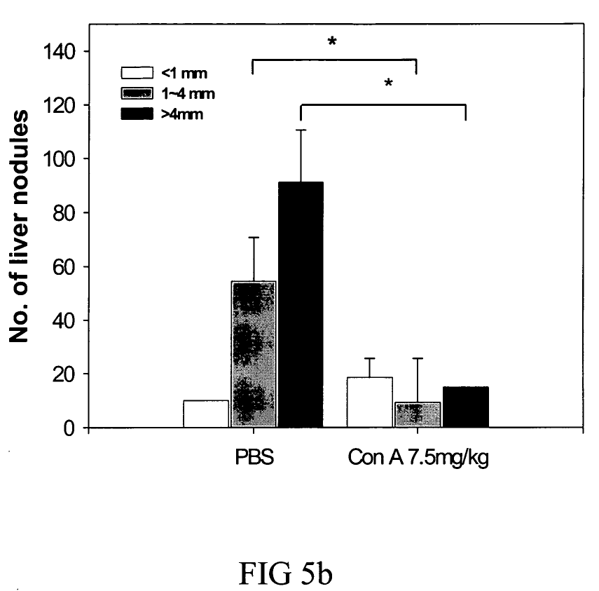

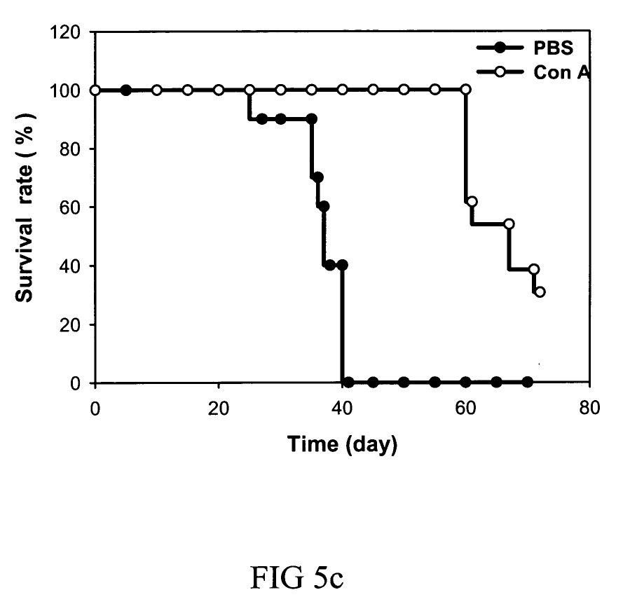

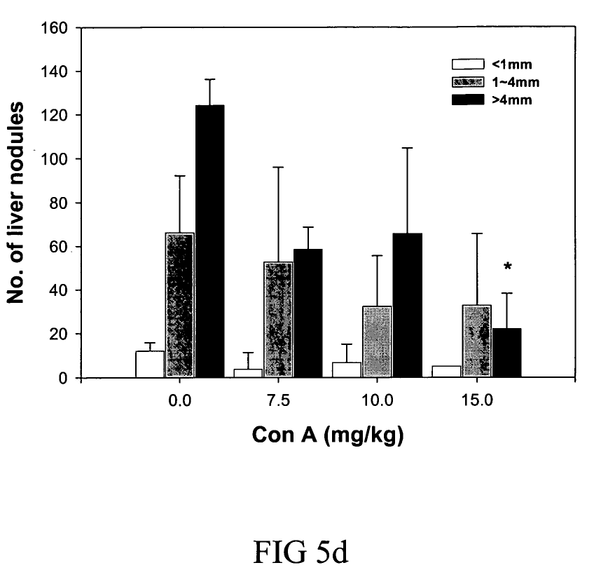

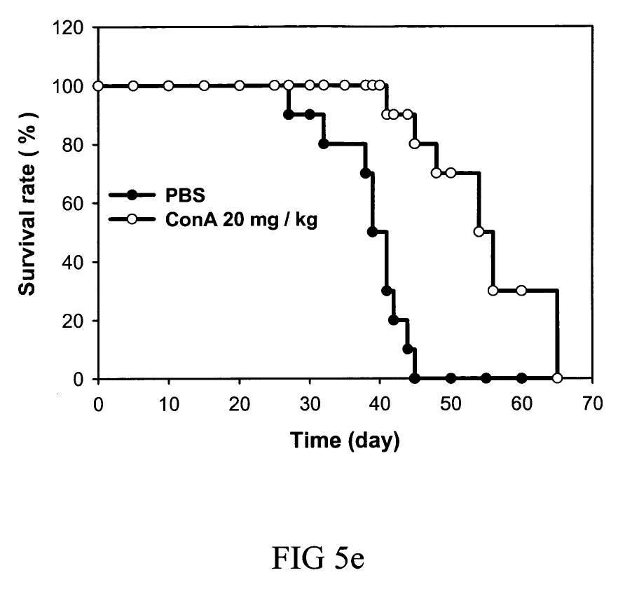

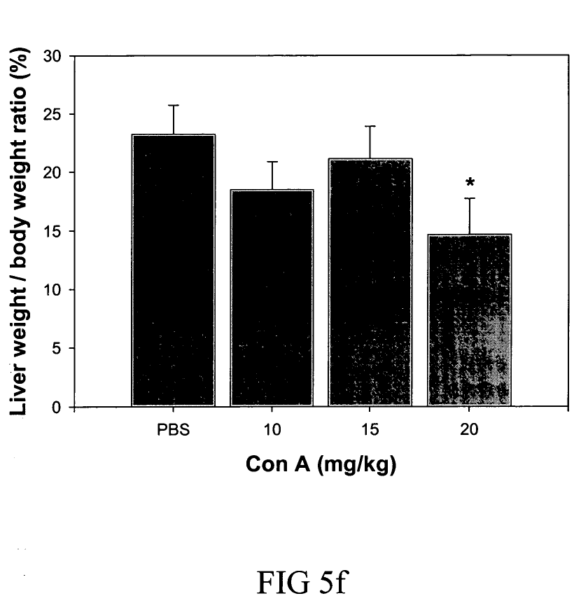

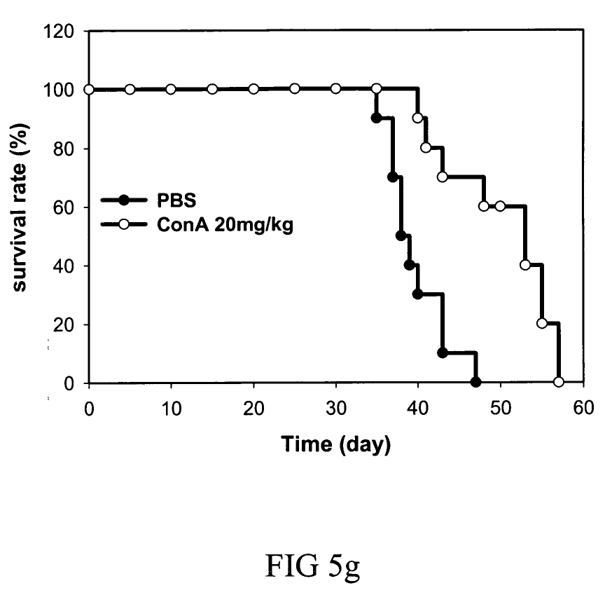

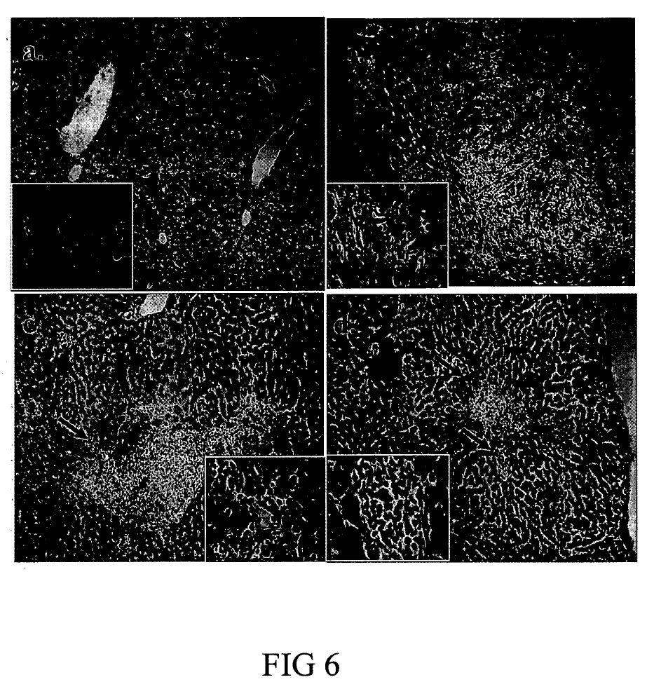

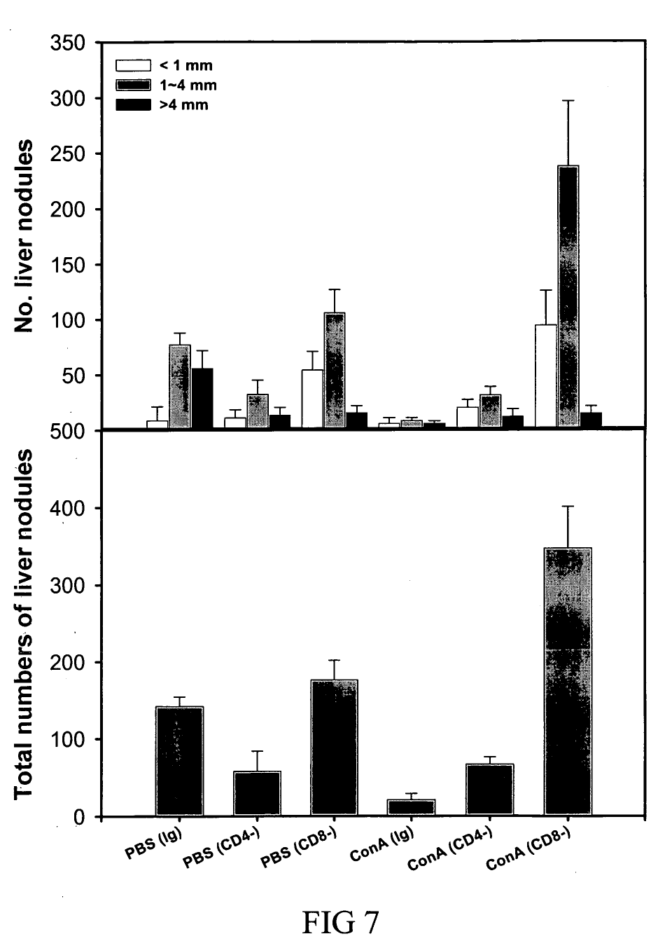

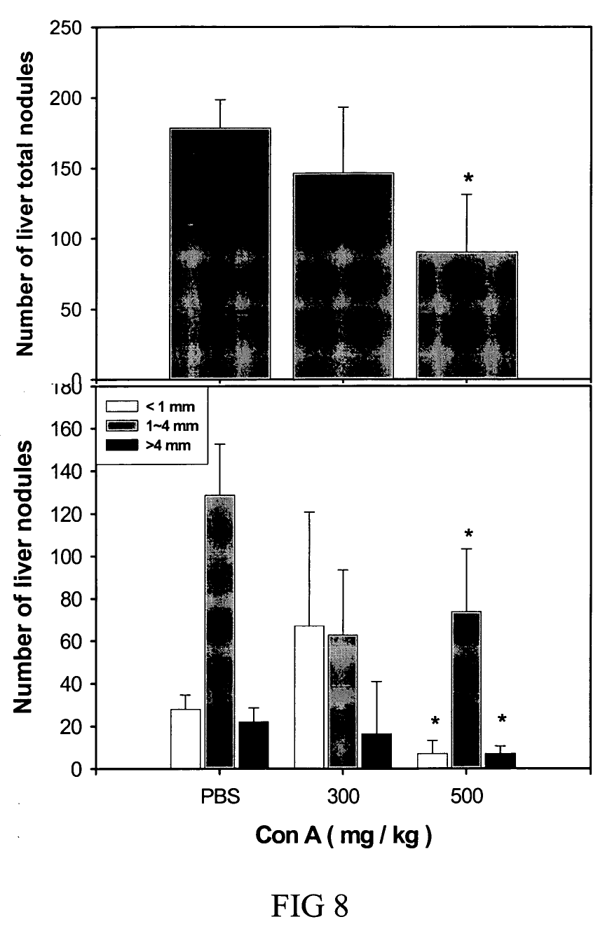

Result: One week after intra-spleen inoculation, the hepatoma-bearing mice were treated intravenously with Con A (7.5 mg/kg body weight) twice at three-day interval. The liver tumor nodule formation was found to be inhibited significantly (FIGS. 5a and 5b). The control mice had 150 tumor nodules of varying sizes while the Con A-treated mice had only 40 tumor nodules. The numbers of large tumor nodules (1-4 mm or >4 mm) decreased dramatically. Around 30%-40% of the mice were tumor-free. In the survival experiment, the survival of the hepatoma-bearing mice was prolonged from 40 to 70 days after Con A treatment, while 20%-30% of the mice were cured (FIG. 5c). When the dose of Con A and the number of injections were increased, for example to 20 mg/kg and 4 times, the liver tumor nodules could be completely eradicated (data not shown). The therapeutic effect of Con A on a large tumor burden was further evaluated. For example, two weeks after intra-spleen inoculation of 1×10⁶ ML-1₄ₐ cells, Con A was injected twice intravenously at doses of 7.5, 10, or 15 mg/kg. The number of liver tumor nodules decreased dose-dependently, especially the large tumor nodules (>4 mm). Statistical significance was observed with the group treated with 15 mg/kg of Con A (p<0.05) (FIG. 5d). In the survival experiment, the dose was increased to 20 mg/kg and Con A was injected 5 times at 3-day intervals. The life span of hepatoma-bearing mice was prolonged from 45 to 65 days (FIG. 5e). The model was further extended to three-week-old, hepatoma-bearing mice, and Con A treatment still had partial effects. In this experiment, the tumor nodules were too numerous to count, so the liver/body weight ratio was used as an index for tumor growth instead. Con A at a dose of 20 mg/kg had significant partial inhibitory effects on tumor growth in the liver (FIG. 5f). The survival of hepatoma-bearing mice was also prolonged for 10 days after Con A treatment (FIG. 5g). This suggests that Con A has some therapeutic effect even on a large liver tumor mass. During the inhibition or eradication of the hepatoma in the liver, many lymphocytic infiltrations were observed in the liver tumor nodule post Con A injection as revealed by the histological tissue staining (FIG. 6). Furthermore, at the dose of 7.5 mg/kg, no elevation of serum AST/ALT in hepatoma-bearing mice was observed, but when the Con A dose was increased to 20 mg/kg, the serum level of AST/ALT was elevated (data not shown). As Con A is known to cause lymphocyte-dependent hepatocyte damage in normal mice, the Con A-activated lymphocyte would infiltrate into the liver to kill the hepatoma cells. The cells participating in this inhibition was demonstrated by the in vivo depletion of CD4⁺ or CD8⁺ T cells. As shown in FIG. 7, the depletion of CD8+T cells blocked the inhibitory effect of Con A, suggesting that CD8⁺ cells played a major role in the Con A-mediated anti-hepatoma activity. The depletion of CD4⁺ T cells also partially affected the Con A anti-tumor activity, but in the no Con A-treated control group, the CD4⁺ T depletion also partially inhibited the tumor formation. To understand whether Con A has any preventive effect on hepatoma by oral feeding, the Con A at 500 mg/kg by oral feeding every day can partially prevent the liver tumor formation (FIG. 8). Lower dose of 300 mg/kg did not show significant inhibition of hepatoma growth.

### EXAMPLE 4

### Con A Inhibited Hepatic Metastasis of Colon Cancer Cell in vivo

Hepatic metastasis model. In the liver metastatic CT-26 model, 1×10⁵ CT-26 cells were inoculated by intra-spleen injection, and the liver nodule formations were counted 21 days post injection. Con A was administrated to mice at different concentrations (mg/kg) via a tail vein as a solution in pyrogen-free DPBS as a volume of 500 μL.

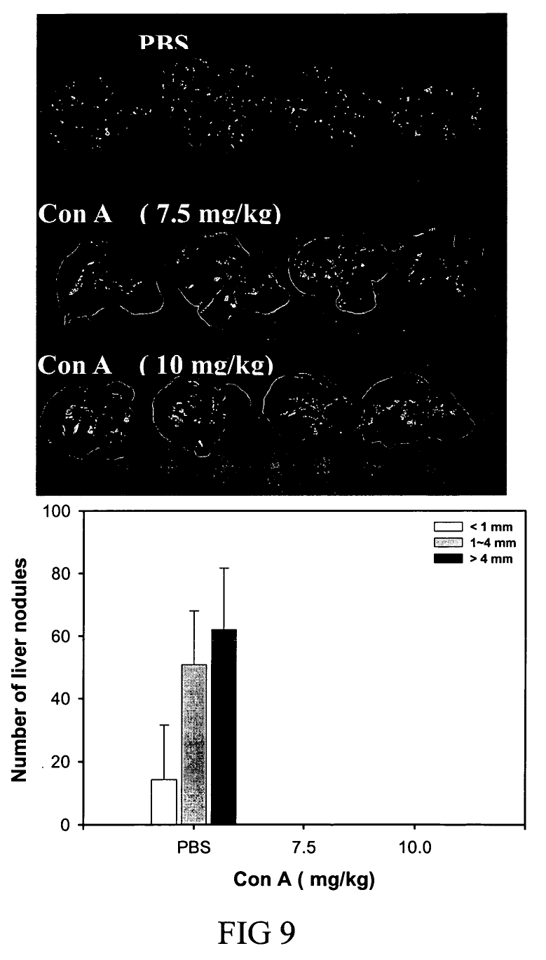

Result: Colon cancer cell line CT-26 was used as a hepatic metastasis model in BABL/c mice to evaluate the anti-metastatic effect of Con A. Con A at doses of either 7.5 or 10 mg/kg completely suppressed the hepatic metastasis of CT-26 into the liver (FIGS. 9a and 9b).

### EXAMPLE 5

### Con A-Induced Eradiation of Tumor Could Prevent Relapse of the Same Tumor

Tumor re-challenge mice model. In the re-challenge model, the tumor-free mice after Con A-induced eradication of the liver tumor were inoculated dorsally subcutaneously with 2×10⁶ ML-1₄ₐ or CT-26. The tumor sizes were measured every three days.

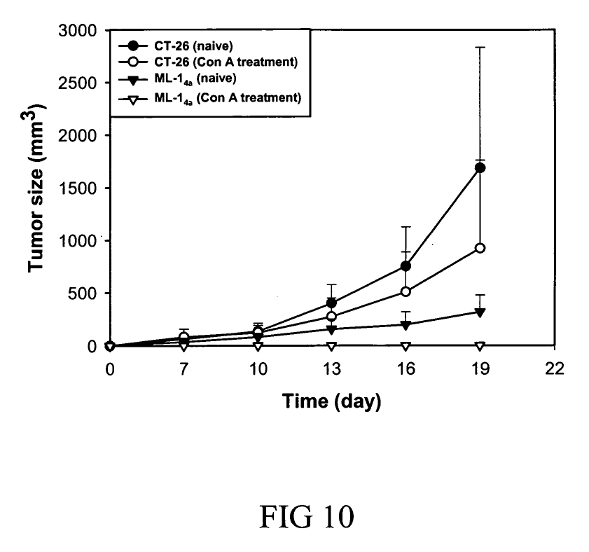

Result: To understand whether Con A-induced eradication of ML-1₄ₐ hepatoma will establish ML-1₄ₐ tumor-specific immunities and prevent the next tumor formation, the tumor-free mice after Con A treatment were inoculated subcutaneously with either ML-1₄ₐ or CT-26 dorsally two months later. ML-1₄ₐ could no longer grow in ML-1₄ₐ-sensitized mice compared with naive mice, but CT-26 tumor cells did grow (FIG. 10), suggesting that tumor-specific memory was established during the early Con A-induced tumor regression.

### EXAMPLE 6

### Result of Con A Inhibiting Liver Tumor Nodule Formation in Immunodeficient Mice

Mice and in situ hepatoma model. Immunodeficient NOD/LtSz-Prkdc<SCID>J mice were provided by the Animal Center of Tzu-Chi University (Hualien, Taiwan), and maintained in the pathogen-free facility of the Animal Laboratory of National Cheng Kung University. In situ hepatoma model was set up by intrasplenic injection of 1×10⁶ viable ML-1₄ₐ cells in 0.1 mL of DMEM into anesthetized mice (Pentobarbital, 50 mg/kg i.p.) and the liver nodule formations were counted 21 days post injection. Con A was administrated to mice at different concentrations (mg/kg) via a tail vein as a solution in pyrogen-free DPBS as a volume of 500 μL.

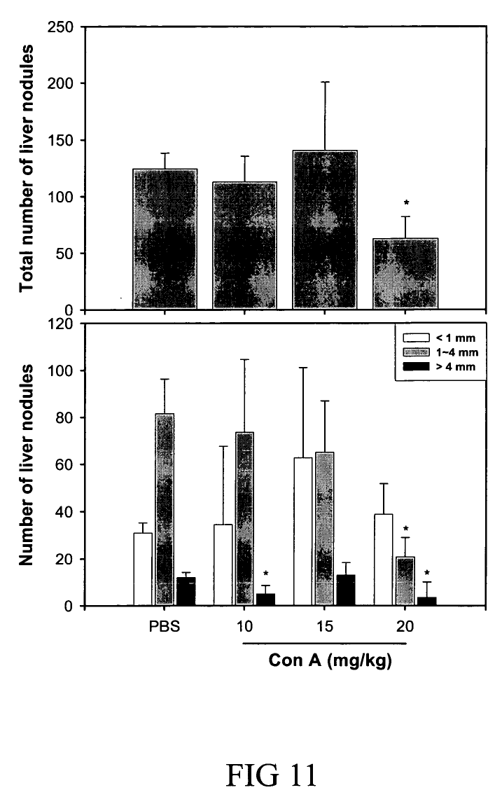

Result: The direct in vivo effect of Con A was tested on liver tumor nodule formation in severe combined immune deficiency (SCID) mice. SCID mice were inoculated with ML-1₄ₐ cells, then 1 week later, treated with Con A at various doses intravenously twice at a 3-day interval. Con A at a dose of 20 mg/kg could inhibit the liver tumor formation in SCID mice (FIG. 11), indicating that Con A has an inhibitory effect on liver tumor nodule formation independently of lymphocyte activation.

### EXAMPLE 7

### Effects of Lectins PSA, LCA or PHA-L Inhibiting Liver Tumor Nodule Formation in vivo

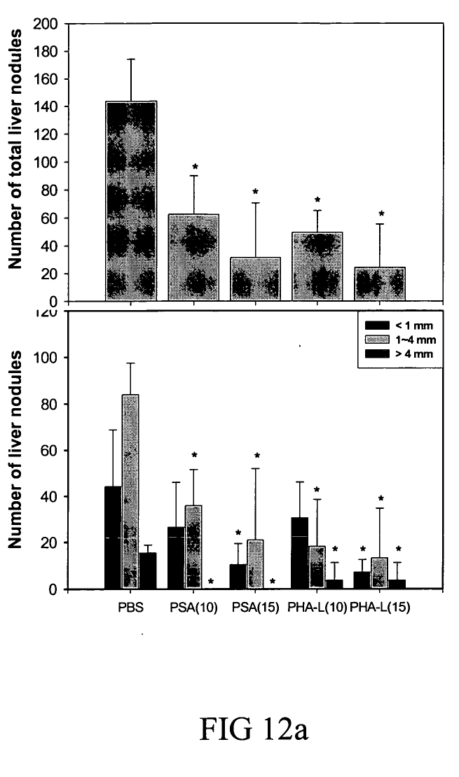

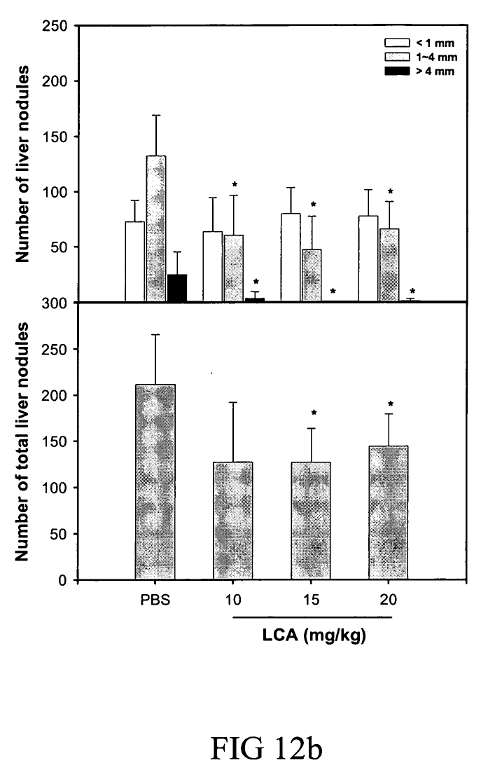

Other lectins for anti-tumor effect in the same model were further explored. Pisum Sativum Agglutinin, Lens Culinaris Agglutinin (LCA) and Phaseolus vulgaris leucoagglutinin from Pisum sativum, Lens culinaris and Phaseolus vulgaris, respectively, were chosen. PSA, LCA and PHA-L could bind ML-1₄ₐ cells specifically and are mitogenic to lymphocytes. PSA, LCA or PHA-L at 10 or 15 mg/kg dose could significantly inhibit ML-1₄ₐ liver tumor nodule formation in BALB/c mice (FIG. 12). The number of the large nodules (>4 mm) decreased especially dramatically.

### EXAMPLE 8

### Differential Binding Assay of Con A, PSA or LCA on Human Hepatocellular Carcinoma Tissue

Immunofluorescent lectin staining on HCC tissue. Paraffin-fixed human HCC tissue was utilized for lectin staining. The sectioned human HCC tissues were stained with 20 μg/mL Con A-FITC, LCA-FITC or PSA-FITC at 4° C. overnight. After wash with PBS 3 times, the tissue sections were incubated with Hoechst for nucleus staining at room temperature for another 15 minutes. The lectin binding to human HCC tissue was observed by fluorescent microscope. The normal part of the same patient was included as control for comparison. Methyl-αD-mannopyranoside (0.5 M) was used to block the specific binding.

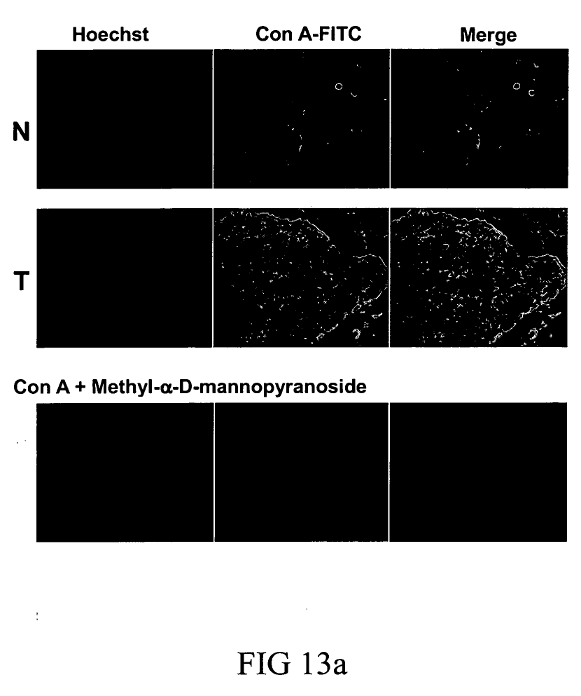

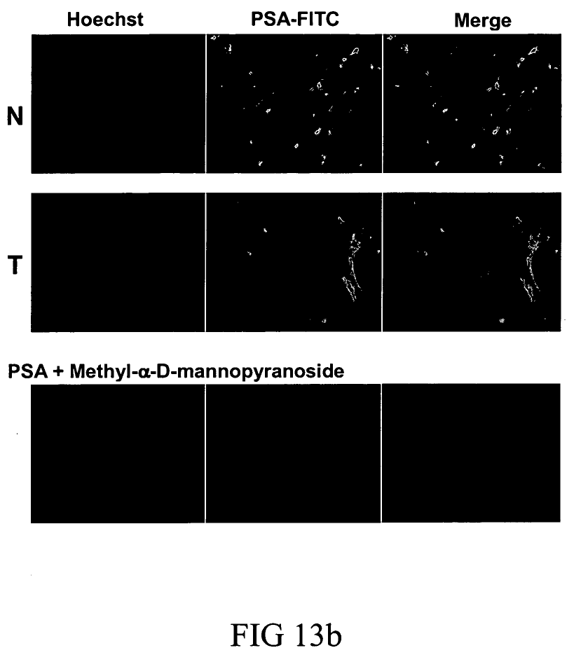

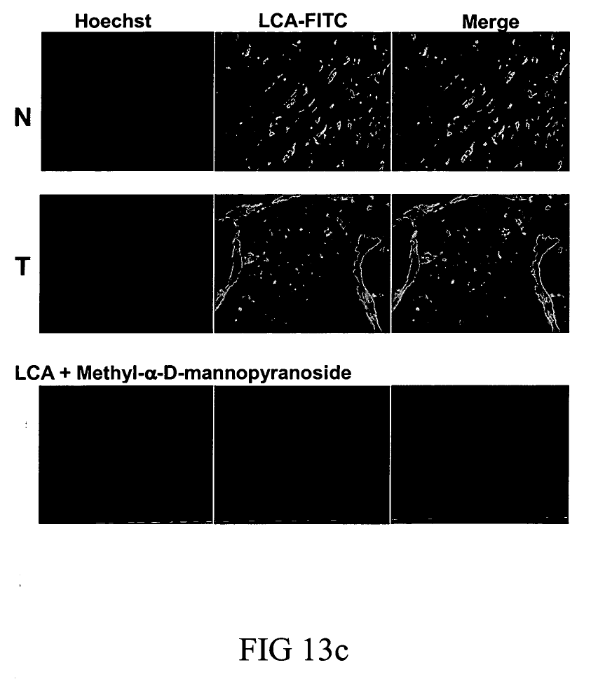

Result: The lectin binding to human hepatoma tissues were further tested by immunofluorescent staining. Con A would bind to normal liver tissue. However, the Con A binding to the tumor part was more intense than normal part of the same patient (FIG. 13a). The differential staining of lectin on normal and tumor tissue was also found for PSA (FIG. 13b) and LCA (FIG. 13c). The Con A, PSA or LCA binding can be blocked by methyl-αD-mannopyranoside, indicating its mannose-specificity. Many HCC patients were tested. The higher binding on tumor part than normal part was always found for Con A, PSA or LCA in the same patient, although a slightly different binding profile (data not shown). HCC might have a different glycoylation patterns that can be differentially bound by lectins of Con A, PSA or LCA.

Summary of lectins on lymphocyte mitogenic activity, cytotoxicity, and anti-tumor effect in vivo is shown in Table 1.

TABLE 1

| | Sugar | Lymphocyte | Tumor cells | Therapeutic |
|---|---|---|---|---|
| Lectin | specificity | mitogenicity | cytotoxicity | effect in vivo |
| Con A | Glucose, | ++++ | ++ | +++ |
| | Mannose | | | |
| PHA-L | Complex | + | + | ++ |
| | structures | | | |
| PSA | Glucose, | +++ | − | + |
| | Mannose | | | |
| LCA | Glucose, | ++ | ++ | + |
| | Mannose | | | |
| WGA | N-acetyl | − | +++ | NT |
| | glucosamine, | | | |
| | Sialic acid | | | |
| RCA-I | N-acetyl | − | +++ | NT |
| | galactosamine, | | | |
| | Galactose | | | |
| GSL-I | N-acetyl | − | − | NT |
| | galactosamine, | | | |
| | Galactose | | | |
| DBA | N-acetyl | − | − | NT |
| | galactosamine | | | |
| SJA | N-acetyl | − | − | NT |
| | galactosamine | | | |
| PNA | Galactose | − | − | NT |
| SBA | Galactose | − | − | NT |

NT, not tested.
*GSL-I, DBA, SJA, PNA, and SBA have carbohydrate-specific binding to cells, but have neither lymphocyte mitogenicity nor tumor cell cytotoxicity.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for treating tumor and/or preventing tumor metastasis and relapse in a subject comprising administrating to the subject a therapeutically effective amount of lectin capable of binding to the tumor and having tumor cytotoxicity and/or lymphocyte activation activity.

2. The method according to claim 1, which is for treating tumor.

3. The method according to claim 1, which is for preventing tumor metastasis.

4. The method according to claim 1, which is for inhibiting tumor relapse.

5. The method according to claim 1, which is for treating tumor and preventing tumor metastasis and relapse.

6. The method according to claim 1, wherein the tumor is a hepatoma.

7. The method according to claim 6, wherein the tumor is a primary hepatoma.

8. The method according to claim 6, wherein the tumor is a metastatic hepatoma.

9. The method according to claim 8, wherein the tumor is a hepatoma transferred from colon cancer cells.

10. The method according to claim 1, wherein the lectin has the ability of inducing tumor apoptosis.

11. The method according to claim 1, wherein the lectin is selected from the group consisting of Concanavalin A (Con A), Pisum Sativum Agglutinin (PSA) and Phaseolus vulgaris leucoagglutinin (PHA-L).

12. The method according to claim 11, wherein the lectin is Concanavalin A.

13. The method according to claim 1, wherein the lectin is administrated by injecting to the tumor directly.

14. The method according to claim 1, wherein the lectin is administrated orally.

15. A composition for treating tumor and/or preventing tumor metastasis and relapse comprising a therapeutically effective amount of lectin capable of binding to the tumor and having tumor cytotoxicity and/or lymphocyte activation activity.

16. The composition according to claim 15, wherein the tumor is a hepatoma.

17. The composition according to claim 16, wherein the tumor is a primary hepatoma.

18. The composition according to claim 16, wherein the tumor is a metastatic hepatoma.

19. The composition according to claim 18, wherein the tumor is a hepatoma transferred from colon cancer cells.

20. The composition according to claim 15, wherein the lectin has the ability of inducing tumor apoptosis.

21. The composition according to claim 15, wherein the lectin is selected from the group consisting of Concanavalin A, Pisum Sativum Agglutinin and Phaseolus vulgaris leucoagglutinin.

22. The composition according to claim 21, wherein the lectin is concanavalin A.

23. The composition according to claim 15, which is in a form of pharmaceutical composition or food composition.

24. The composition according to claim 15, which is administrated by injecting to the tumor directly.

25. The composition according to claim 15, which is administrated orally.
